(19) United States
(12) Patent Application Publication
(10) Pub. No.: US 2013/0310665 A1
(43) Pub. Date: Nov. 21, 2013

(54) METHOD

(71) Applicants: Abina M. Crean, Cork (IE); Marie G. McGrath, Cork (IE); Conor O'Mahony, County Kerry (IE); Anto Vrdoljak, Sesvete (HR); Anne C. Moore, Cork (IE); John B. Carey, Cork (IE)

(72) Inventors: Abina M. Crean, Cork (IE); Marie G. McGrath, Cork (IE); Conor O'Mahony, County Kerry (IE); Anto Vrdoljak, Sesvete (HR); Anne C. Moore, Cork (IE); John B. Carey, Cork (IE)

(73) Assignee: UNIVERSITY COLLEGE CORK, CORK (IE)

(21) Appl. No.: 13/885,372

(22) PCT Filed: Nov. 18, 2011
(86) PCT No.: PCT/IB11/55175

§ 371 Date: Aug. 6, 2013

Related U.S. Application Data
(60) Provisional application No. 61/458,167, filed on Nov. 18, 2010.

(30) Foreign Application Priority Data
Nov. 18, 2010 (GB) 1019577.4

(51) Int. Cl. A61K 9/70; A61B 5/145

(52) U.S. Cl. CPC A61K 9/703; A61B 5/14503
USPC 600/309; 264/299; 427/2.31

(57) ABSTRACT

The present invention relates to a method for fabricating a microneedle which comprises the steps of spraying a composition into a mould, drying the composition and removal of the dried composition from the mould, thereby forming a microneedle that, when applied to the skin of a subject, pierces the stratum corneum to access the underlying tissue of the subject. The present invention also relates to a method for coating a microneedle which comprises the steps of spraying a composition onto a microneedle and drying the composition at an ambient temperature, thereby forming a coated microneedle that, when applied to the skin of a subject, pierces the stratum corneum to deliver the sprayed material to the underlying tissue of the subject.

### FIELD OF THE INVENTION

The present invention relates to a method for making a microneedle that, when applied to the skin of a subject, pierces the stratum corneum to access the underlying tissue of the subject. In particular, it relates to a method which comprises the step of spraying a composition (1) on to the surface of a microneedle array or (2) into a mould to form at least part of the microneedle.

### BACKGROUND TO THE INVENTION

Vaccines may be administered through various routes of delivery, including oral, nasal, intramuscular (IM) or intradermal (ID). While vaccination represents the primary public health measure to combat infectious diseases, it suffers from poor compliance, potential for nocosomial infections and logistical obstacles of cost, stability, storage, distribution and disposal of used sharps. Development of needle-free, painless, safe, efficacious immunization strategies is an important goal in global health care.

An alternative delivery system that is useful for some drugs is the transdermal patch, which relies on diffusion of the drug across the skin for delivery. However this delivery option is only viable for a small subset of low molecular weight, lipophilic drugs due to the effective barrier properties of the skin.

Transdermal drug delivery or intradermal drug delivery allows for simpler administration of drugs, altered bioavailability and pharmacokinetic profiles.

Microneedle arrays have been proposed as a hybrid between hypodermic needles and transdermal patches to address the limitations of both existing technologies. Microneedles are solid or hollow arrays of micron scale projections ranging in height typically from 50-700 μm or more.

Microneedle arrays can facilitate the passage of materials, including drugs, through or into human skin and other biological membranes in circumstances where ordinary transdermal administration is inadequate. Microneedle arrays can also be used to sample fluids found in the vicinity of a biological membrane, such as interstitial fluid, which is then tested for the presence of biomarkers.

### Micromoulding

Microneedles may be made by micromoulding, by providing a mould having a microdepression which defines the surface of the microneedle, filling the microdepression with moulding material and moulding the material to form a microneedle. For example, the micromould may be filled with liquid monomer, the monomer is polymerised, and the polymer is directly or indirectly converted to a solid form in the shape of the microneedle. The material of interest can be included in the composition of the moulded microneedles.

However, methods involving the use of liquids (molten liquids, solutions and suspensions) suffer various drawbacks associated with surface tension and viscosity effects of the liquids being filled into moulds.

When filling moulds, poor wetting is usually most pronounced at the tip of microneedle mould and results in poorly formed or crooked microneedle tips. This can produce microneedles that are not mechanically strong and are incapable of penetrating the stratum corneum. A second issue related to viscosity is associated with liquid moulding is the presence of bubbles in the filled moulds which causes the formation of void defects in the microneedles, undermining their structural integrity.

Various strategies have been proposed to address these issues, including the use of vacuum to remove entrapped bubbles and help pull the polymer melt into the mold (Park et al (2005) Journal of Controlled Release 104 (1): 51-66). An alternative approach is to mould microneedles from concentrated hydrogels by centrifuging the filled mold at 3000×g to fill the microneedle mould (Lee et al. 2008 Biomaterials 29 (13): 2113-2124).

However, neither of these approaches is suitable for large-scale moulding operations. Both approaches would require the development of specialised equipment and the inclusion of several extra steps into the manufacturing process. It is desirable for microneedle manufacturing methods to be inexpensive, for example so that it is financially feasible for microneedle arrays to be marketed as disposable devices. A disposable device is preferable to a reusable one as it avoids the question of the integrity of the device being compromised by the previous use and the potential need to resterilize the device after each use.

There is thus a need to provide an improved method for fabricating microneedles from liquids that avoids the disadvantages associated with known methods.

### Spray-Coating

Microneedle arrays may be fabricated from metals, silicon, silicon dioxide, biodegradable polymers as well as other materials. Silicon microneedles can be fabricated using deep-reactive ion-etching (DRIE) or wet-etch technology as described, for example, in U.S.2007/0134829A1. The microneedle arrays may be coated with a material of interest.

Methods for coating microneedles to form solid drug containing formulations have been described previously. Current state of art in the coating of microneedles involves the use of specialised coating apparatus for dip coating or immersing the microneedle array in desired formulations (Prausnitz WO20006/138719); rolling (Trautman WO2002/074173) or brushing on the formulation (WO2008139648); or pattern coating using, for example, ink jet coating or microfluidics (Cormier U.S.2009/0186147) that require the use of wetting agents. Trautman (WO2002/074173) describes a method of coating a liquid on microprojections without coating the liquid on the substrate using a roller, and immersing microprojections to a predetermined level. Gill et al., (Journal of Controlled Release, 2007, 117, 227-237), describes a process for coating microneedles via micro dip-coating in a reservoir containing a cover to restrict the access of liquid only to the microneedle shaft. Both of these methods rely on varying the number of contacts (dips) between the microneedle and the reservoir or roller to control a dosage of biologically active compound to be coated on the microneedle. PCT Application No. PCT/US06/23814 also describes methods for coating of microneedles to form solid drug containing formulations by multiple contacts between the microneedle and the coating liquid. The use of masking fluids has also been described (for example in WO2007/059289 and WO2007/061964). The masking fluids are based on organic compounds that are more volatile than the coating fluid, to mask the base of the array from the coating formulation which is added on top, this results in coated microneedle tips.

With respect to coating material onto a solid microneedle, the micrometre lengths of the microneedles and their close proximity to each other impose challenges on how to uniformly and efficiently coat these devices. This is largely due to the significant effects of surface tension, capillarity and viscous forces at these micron scales (Gill and Prausnitz, Pharm. Res. 2007, 24, 1369-80). Surface tension can cause limited wetting of surfaces. When coating microneedles, poor wetting can result in poor coalescence of liquid which is required to form an intact film. Viscosity of liquids can influence how liquids flow on microneedles surfaces when coating microneedles and impact on film formation.

Spray coating techniques have also been described, for example in WO2009/081125. However, this technology requires elevated temperatures and heated aerosols to form a stable coat subsequent to spraying. This limits range of pharmaceutical compounds that can be applied to the microneedles using this technology as it is unsuitable for thermolabile compounds and live entities, such as viruses, bacteria etc for use in prophylactic or therapeutic vaccination or gene therapy.

There is thus a need for improved methods for coating microneedles which address the issues of viscosity and surface tension and are suitable for use with thermolabile materials.

### SUMMARY OF ASPECTS OF THE INVENTION

The inventors have surprisingly found that the problems associated with surface tension associated with liquid microneedle forming methods may be overcome by using a method which comprises the step of spraying a microneedle-forming composition into the microneedle mould.

Spray-formation of microneedles also facilitates the fabrication of microneedles having two or more layers, for example with different structural or pharmacological properties.

Thus, in a first aspect, the present invention provides a method for fabricating a microneedle which comprises the steps of spraying a composition into a mould, drying the composition and removal of the dried composition from the mould, thereby forming a microneedle that, when applied to the skin of a subject, pierces the stratum corneum to access the underlying tissue of the subject.

The method is particularly suited for forming dissolvable microneedles, by using a composition which forms a dissolvable material following drying, such that when the microneedle is applied to the skin of a subject it dissolves.

The composition may comprise an agent, such as a pharmaceutical, vaccine or diagnostic agent. For example, the agent may be dispersed in a solution which forms a dissolvable material following drying, such that when the microneedle is applied to the skin of a subject it dissolves, causing delivery of the agent into the underlying dermal tissue of the subject.

The method may involve drying the composition at ambient temperature. This particularly advantageous for use in connection with thermolabile agents.

The method may comprise a plurality of successive spraying and drying steps to create a microneedle having a plurality of layers.

Alternatively, the method may involve a single spraying step to create the microneedle.

Where the method comprises a plurality of successive spraying and drying steps, the steps may involve application of a plurality of different compositions which comprise different agents or different materials which may form layers having different properties, such as strength, permeability or dissolvability.

Suitable dissolvable materials include: polymers, carbohydrates, cellulosics, sugars, polyols or alginic acid or a derivative thereof and any material that preserves the chemical and physical stability of the active agent.

The dissolvable material may be one or a combination of materials such as: polyvinyl alcohol (PVA), polyvinyl pyrrolidone (PVP), raffinose, sucrose, trehalose, glycerine, CMC and sodium alginate.

The agent may be a therapeutic, prophylactic or diagnostic agent. The agent may be a drug or vaccine.

The agent may, for example, be selected from the following group: an antibody, a live or inactivated virus or viral vector, a bacterium, protein, glycoprotein, lipid, oligosaccharide, polysaccharide, nucleotides, oligonucleotides, DNA or RNA.

The microneedle-forming composition may comprise a vaccine adjuvant such as trehalose or other sugars or oligonucleotides (e.g. polyI:C).

The method of the invention may be used to make a microneedle comprising an outer layer and an inner layer, in which method the outer layer is made by spraying a composition into a microneedle mould. The inner layer may be fabricated using a non-spraying method, such as pour-filling the remainder of the apertures with inner layer-forming composition.

In a second aspect the present invention provides a microneedle fabricated by the method of the first aspect of the invention, in particular a method for making a microneedle comprising an outer layer and an inner layer, in which method the outer layer is made by spraying a composition into a microneedle mould.

The microneedle may be fabricated such that the outer layer dissolves before the inner layer following application of the microneedle to the skin of a subject.

The microneedle may be fabricated such that the outer layer acts as a moisture barrier; a light barrier; a barrier to oxidation or other degradative chemical reactions; and/or a barrier for handling to protect the user from a toxic inner layer.

The outer layer may comprise a rapid dissolving excipient and the inner layer may comprise a slow release excipient.

The outer and inner layers may contain incompatible materials that are not in contact with each other. In a related embodiment, the outer and inner layers may be separated by a third layer that divides the incompatible materials.

In order to increase skin-piercing capacity, the outer layer of the microneedle may form a tip which is made from a material of high mechanical strength and the remainder of the microneedle may be made from a material of low mechanical strength.

Alternatively the outer layer may be made from a material of low mechanical strength and the internal layer made from a material of high mechanical strength.

For diagnostic applications, the outer layer may comprise a slow dissolving or porous or non-dissolving excipient and the inner layer may comprise a composition for sampling body fluids, such that the outer layer acts as a cage for the material of the inner layer. The composition may comprise a sampling agent, such as an antibody; a substance that can conduct a signal; or a substance for monitoring iron content, or red blood cell concentration or shape or other biological matter, or for monitoring interstitial fluid.

The outer layer may comprise an amphiphilic material.

In a third aspect, the present invention provides a microneedle array comprising a plurality of microneedles according to the second aspect of the invention.

The invention also provides a method for delivering an agent to a subject, which comprises the step of applying an array according to the third aspect of the invention which comprises the agent coated on or dispersed in at least one layer of the microneedles, such that the agent is delivered to the underlying tissue of the subject.

The invention also provides a method for absorbing a material from a subject which comprises the step of applying an array comprising a plurality of microneedles according to the second aspect of the invention to the skin of the subject such the material absorbs to the sampling composition of the inner layer.

The method for fabricating a microneedle according to the first aspect of the invention may comprise the step of application of a backing layer to the mould, once filled. The backing layer may be (i) of high mechanical strength, (ii) inert and/or (iii) made of non-degrading material.

The method may also comprise the step of applying an adhesive layer, either as or on top of a backing layer.

The method of the first aspect of the invention may be used for making a microneedle array comprising a plurality of microneedles, for example by comprising the step of spraying a composition into a mould which comprises a plurality of microneedle-forming apertures.

The invention also provides a kit for use in a method according to the first aspect of the invention, which comprises a composition for spraying into a mould.

The kit may also comprise one or more of the following:

a microneedle-forming mould;

spraying apparatus; and/or

a backing and/or adhesive layer.

The invention also provides a device comprising a microneedle or microneedle array according to the second or third aspects of the invention.

The inventors have also surprisingly found that the problems associated with surface tension associated with known microneedle coating methods may be overcome by using a method which comprises the step of spraying a coating-forming composition on to the microneedle.

Thus, in a fourth aspect, the present invention provides a method for coating a microneedle which comprises the steps of spraying a composition onto a microneedle, drying the composition at an ambient temperature, thereby forming a coated microneedle that, when applied to the skin of a subject, pierces the stratum corneum to deliver the sprayed material to the underlying tissue of the subject.

The spray-coating composition may comprise a thermolabile entity. The spry-coating composition may comprise a live agent such as a bacterium or virus.

The method may involve preferentially spray-coating the shaft of the microneedle such that the microneedle tip retains its sharpness. It is possible to alter the pattern of composition distribution between the microneedle tip, shaft and base of the microneedle array by altering the rate of liquid input into the nozzle and/or the composition of the sprayed mixture (see examples).

Spray-coating of microneedles also facilitates the fabrication of microneedles having two or more layers, for example with different structural or pharmacological properties.

The composition may comprise an agent, such as a pharmaceutical, vaccine or diagnostic agent. For example, the agent may be dispersed in a solution which forms a coating material following drying, such that when the microneedle is applied to the skin of a subject it causes delivery of the agent into the underlying dermal tissue of the subject.

The spraying composition may comprise one or more stabilising excipients such as amorphous glasses.

The spraying composition may be substantially free from viscosity enhancers and/or film-forming agents which may affect drug and/or virus stability.

The method may comprise a plurality of successive spraying and drying steps to create a microneedle having a plurality of layers.

Alternatively, the method may involve a single spraying step to create the coated microneedle.

Where the method comprises a plurality of successive spraying and drying steps, the steps may involve application of a plurality of different compositions which comprise different agents or different materials which may form layers having different properties, such as strength, permeability or dissolvability.

The agent may be a therapeutic, prophylactic or diagnostic agent. The agent may be a drug or vaccine.

The agent may, for example, be selected from the following group: an antibody, a live or inactivated virus or viral vector, a bacterium, protein, glycoprotein, lipid, oligosaccharide, polysaccharide, nucleotides, oligonucleotides, DNA or RNA.

The microneedle-coating composition may comprise a vaccine adjuvant such as trehalose or other sugars or oligonucleotides (e.g. polyI:C).

The method of the invention may be used to make a microneedle comprising an outer coating layer and an inner coating layer, in which method the outer layer is made by spraying a composition on to a microneedle.

In a fifth aspect the present invention provides a microneedle fabricated by the method of the fourth aspect of the invention, in particular a method for coating a microneedle comprising an outer layer and an inner layer, in which method the outer layer is made by spraying a composition on to a microneedle.

The microneedle may be fabricated such that the outer layer acts as a moisture barrier; a light barrier; a barrier to oxidation or other degradative chemical reactions; and/or a barrier for handling to protect the user from a toxic inner layer.

The outer layer may comprise a rapid dissolving excipient and the inner layer may comprise a slow release excipient.

The outer and inner layers may contain incompatible materials that are not in contact with each other. In a related embodiment, the outer and inner layers may be separated by a third layer that divides the incompatible materials.

In order to increase skin-piercing capacity, the outer layer (i.e. coating) of the microneedle may form a tip which is made from a material of high mechanical strength and the remainder of the microneedle may be made from a material of low mechanical strength.

Alternatively the outer layer may be made from a material of low mechanical strength and the internal layer made from a material of high mechanical strength.

For diagnostic applications, the outer layer (i.e. coating) may comprise a slow dissolving or porous or non-dissolving excipient and the inner layer may comprise a composition for sampling body fluids, such that the outer layer acts as a cage for the material of the inner layer. The composition may comprise a sampling agent, such as an antibody; a substance that can conduct a signal; or a substance for monitoring iron content, or red blood cell concentration or shape or other biological matter, for monitoring interstitial fluid.

The outer layer may comprise an amphiphilic material.

In a sixth aspect, the present invention provides a microneedle array comprising a plurality of microneedles according to the fifth aspect of the invention.

The invention also provides a method for delivering an agent to a subject, which comprises the step of applying an array according to the sixth aspect of the invention which comprises the agent coated on or dispersed in at least one layer of the microneedles, such that the agent is delivered to the underlying tissue of the subject.

The invention also provides a method for absorbing a material from a subject which comprises the step of applying an array comprising a plurality of microneedles according to the sixth aspect of the invention to the skin of the subject such the material absorbs to the sampling composition of the inner layer.

The method of the fourth aspect of the invention may be used for making a microneedle array comprising a plurality of microneedles, for example by comprising the step of spraying a composition on to a microneedle array comprising a plurality of microneedles.

The invention also provides a kit for use in a method according to the fourth aspect of the invention, which comprises a composition for spraying on to a microneedle or microneedle array.

The kit may also comprise one or more of the following:
a microneedle or microneedle array; and/or
spraying apparatus.

The invention also provides a device comprising a microneedle or microneedle array according to the fifth or sixth aspects of the invention.

The method of the fourth aspect of the invention uses an atomising spray system to coat microneedle arrays which can minimize the influence of viscosity and surface tension. This method of spray coating at ambient temperatures is easily scalable, in comparison to dip coating. As the coating is applied and dried at an ambient temperature, it can be utilised with a wide range of pharmaceutical compounds, including those that are thermolabile in addition to live entities, such as viruses, bacteria etc for use in prophylactic or therapeutic vaccination or gene therapy.

### DESCRIPTION OF THE FIGURES

FIG. 1. Scanning electron microscopy image of an inverted PDMS mould. In the examples used, a mould was generated from an array consisting of 81 microneedles that were 280 μm in height.

FIG. 2. Scanning electron microscopy images of cellulose dissolvable microneedles; A) a single microneedle on the array prepared and B) a number of microneedles on the array prepared. The microneedle array was prepared by filling the mould with a single spray of low viscosity sodium carboxymethylcellulose (5% w/v) and glycerine (0.1% v/v) solution into a PDMS mould, application of a backing layer of the same solution and drying at room temperature prior to removal from the mould.

FIG. 3. Scanning electron microscopy images of dissolvable microneedles prepared from a range of dissolvable materials; PVA microneedle array, PVP microneedle array, raffinose microneedle array and sodium alginate microneedle array with CMC-glycerine backings.

FIG. 4. Fluorescent microscopy images of microneedles showing layers of red fluospheres and green fluorescein; (A) shows two layers; red fluospheres present in the outer layer of the microneedle and fluorescein in the inner section and base. (B) shows three layers; red fluospheres presence in the microneedle tip, a non-fluorescent trehalose layer below and fluorescein present in the base. These images are composed of individual fluorescent images merged using Adobe photoshop.

FIG. 5. Light microscopy images of trehalose microneedles coated or uncoated with Lipoid PG 18:0/18:0 after preparation (Time 0) and after storage at 40° C. and relative humidity of 75% for 72 hours.

FIG. 6. Plot moisture uptake quantified by percentage increase in weight, observed in trehalose microneedles stored at 40° C. and a relative humidity of 75% over 72 hours.

FIG. 7. Light microscopy analysis of pig skin cryosections following application of microneedles. (A) a typical indentation without rupture; (B) a stratum corneum rupture; and (C) an epidermal breach.

FIG. 8. Total number of SC ruptures (A) and epidermal breaches (B) per skin section due to insertion of dissolvable microneedle fabricated from the indicated material. Plot demonstrates the mean and 10^{th} to 90^{th} percentile. (A) The number of SC ruptures was significantly higher in all microneedle groups compared to control (p<0.001). (B) *p<0.05; **p<0.01, ***p<0.001 compared to control, untreated sample, as determined by ANOVA, n=45 sections per group.

FIG. 9. Scanning electron microscopy images of Carboxymethylcellulose sodium (3% w/v) and Tween 80 (1% v/v) aqueous solution spray coated onto a silicon microneedle wafer. The distance from the nozzle to the wafer was 6 cm.

FIG. 10. Scanning electron microscopy images of Trehalose (15% w/v) and Tween 80 (1% v/v) aqueous solution spray dried onto a silicon wafer. The distance from the nozzle to the wafer was 5 cm.

FIG. 11. Scanning electron microscopy images of Trehalose (15% w/v) and Tween 80 (1% v/v) aqueous solution spray dried onto a silicon wafer. The distance from the nozzle to the wafer was 5 cm.

FIG. 12. Scanning electron microscopy images of CMC (1% w/v),trehalose (15% w/v) and Tween 80 (1% v/v) aqueous solution sprayed onto a silicon microneedle array. The distance from the nozzle to the wafer was 5 cm. The liquid input was at 1 ml/5.5 seconds.

FIG. 13. Spray coating results in even distribution of the mixture around the microneedles compared to dropping the mixture onto the microneedle array. A solution of 15% Trehalose, 0.5% Tween and FITC was either pipetted onto a microneedle array (A) or spray coated onto an array (B and C). After drying at ambient temperature, all arrays were imaged by fluorescent microscopy.

### DETAILED DESCRIPTION

### Transdermal Delivery

The function of the skin is to protect against water loss and act as the first line of defence against the entry of pathogens into the body. Mammalian skin can be subdivided into three layers; the stratum corneum (SC); in humans this is 10-20 μm in depth, the viable epidermis (50-100 μm in humans) and the dermis (1-3 mm in humans) The stratum corneum is composed of closely packed dead keratinocytes embedded in a highly organized intercellular lipid matrix that forms a barrier that is impermeable to microbes and large molecules such as vaccine antigens. It is this outer layer that restricts successful transdermal delivery.

A rich network of innate immune cells, such as Langerhans cells (LCs), monocytes and dermal dendritic cells (DC), reside in the underlying epidermis and dermis. Intradermal vaccination with needle and syringe (ID) can induce quantitatively or qualitatively superior immunity compared to intramuscular (IM) or subcutaneous (SC) delivery; this has been exemplified in particular for antibody-inducing influenza vaccines.

The term “transdermal delivery” used herein includes percutaneous delivery.

### Microneedles

A microneedle array or patch is a device for delivering an agent through the stratum corneum of the skin, which comprises a substantially flat base plate, on which is mounted a plurality of microneedles. Upon application to the skin, the microneedles extend through the stratum corneum, and either into the epidermis, or into the underlying dermis.

The microneedle array may be applied to the skin in a single rolling motion, or by simply pushing the patch substantially vertically on to the skin, as described in Haq et al ((2009) Biomed Microdevices 11:35-47).

The microneedle has sufficient mechanical strength to penetrate the stratum corneum.

### Patch Characteristics

The microneedle patch or array may be provided as a discrete patch, for example of between 3-10 mm×3-10 mm in size. Alternatively the patch may form part of a large area (such as a roll) of material, which is subsequently cut to the required size.

The microneedles may be any shape which is suitable for piercing the skin. They may, for example may be tapered, coming to a point at one end for skin piercing. The microprojections may, for example, be substantially conical or pyramidal in shape.

The number of microneedles per patch may range from 10-200, for example 15-100 per patch.

### Dissolvable Material

The microneedles of the present invention may be made at least partly from a dissolvable material which dissolves following application of the array to the skin. Specifically the material dissolves on contact with moisture in the epidermal and/or dermal layers.

An advantage of using dissolvable microneedles is that it eliminates the problem of clinical waste hazards. It is also amenable to slow or episodic release and sampling applications.

Suitable materials include polymers, carbohydrates, cellulosics, sugars, polyols or alginic acid or a derivative thereof.

The dissolvable material may comprise one or a combination of materials, for example selected from the following: polyvinyl alcohol (PVA), polyvinyl pyrrolidone (PVP), raffinose, sucrose, trehalose, glycerine, CMC and sodium alginate.

The microneedle may dissolve completely, such that all of the material of the needle is absorbed by the skin. Alternatively it may partially dissolve such that only part of the material of the microneedle is absorbed by the skin, as long as it still causes delivery of the pharmaceutical agent to the underlying tissue.

The composition for forming the dissolvable material may include one or more stabilising excipients such as amorphous glasses (sugar, carbohydrate and polymer) and surfactants.

### Spraying Method

The method of first aspect of the invention involves spraying a composition into a mould in order to form one or more layers. The method of the fourth aspect of the invention involves spraying a composition on to the surface of a microneedle in order to form one or more layers.

The atomised spray may be created using compressed gas, such as compressed air or nitrogen. A material (such as a pharmaceutical, vaccine or diagnostic agent) to be layered into the microneedle moulds or on to a microneedle may be dissolved or dispersed in a liquid vehicle. The liquid mixture may then be feed into the atomising nozzle where it is mixed with a stream of compressed gas. A suitable nozzle is a Schlick nozzle 970 S8, or equivalent with an orifice of between 0.1 and 1 mm, such as about 0.5 mm.

The atomised spray pattern may comprise of a circular cone of about 10° to 40° or an oval flat spray of about 30°×70°. The droplets comprising the spray range from a fog-like spray to very fine droplets. For use in the method of the first aspect of the invention, the droplets produced should be small enough to fill the tips of the microneedle mould without forming air bubbles, in order to form sharp-tipped needles. The average droplet size may be less than 15×, 10× or 5× radius of the microneedle tip. For a microneedle tip having a 1 μm radius, the average droplet size may, for example be 15, 10 or 5 μm.

The average size of the droplets may, for example, be less than 15, less than 10 or less than 8 microns in diameter.

Pharmaceutical spraying processes are known for coating tablets or beads with protective and/or drug-containing coatings. The droplet size produced in such processes are at least 20 μm in size (Aliseda et al (2008) Int J Multiphase Flow 34:161-175) and commonly much larger than that. These processes are therefore unsuitable for use with the present invention, but may be modified to be useful by taking steps to reduce the average droplet size.

The spraying temperature may be controlled by fitting the nozzle with a heating/cooling sheath.

In a preferred embodiment, the operation is performed at an ambient temperature as many pharmaceutical agents are unstable or degrade at high temperatures.

The viscosity and surface tension of the sprayed liquid systems may range from 6-350 cP and 36-71 mN·m⁻¹. Excipients for spraying into moulds may, for example, include carboxymethylcellulose, hydroxypropylmethylcellulose, polyvinyl alcohol, polyvinylpyrollidone, sodium alginate, tween 80, glycerine, trehalose, fructose, sucrose, raffinose.

In the method of the first aspect of the invention, the microneedle moulds may be passed under the spray. The distance from the nozzle to the mould can vary depending on the area being sprayed. The distance may be at least 3.5 cm. The thickness of the layer depends on the concentration of material being sprayed, the duration of spraying and area being sprayed.

After spraying, the layer is dried preferably at an ambient or sub-ambient temperature, for example less than 35° C., such as between 10-25 ° C. The duration the drying is variable, and for example can range from 10 minutes to 24 hours. Drying may be performed in a low humidity environment.

In the method of the fourth aspect of the invention, the composition may be dry following a drying step at an ambient temperature for one hour or less, for example 30 minutes or fewer.

### Mould

The method of the first aspect of the invention may also involve making a microneedle mould. The mould may, for example be a female mould constructed from a male master microneedle array. The mould may be made from silicon, metal or polydimethylsiloxane.

### Backing Layer

After the microneedles or microneedle array has been made, a backing layer, for example of flexible polymer, may be applied to facilitate handling and application of the microneedles to the skin.

The backing layer may be of high mechanical strength, inert and/or made of non-degrading material.

The method may also comprise the step of applying an adhesive layer to the filled mould, either as a backing layer or on top of a separate backing layer.

### Layering

The methods of the first and fourth aspects of present invention may involve a plurality of successive spraying and drying steps to create a plurality of layers. The successive spraying and drying steps may involve application of a plurality of different compositions which comprise different agents or different materials.

The order in which the layers are applied can be tailored to optimise mechanical strength stability or pharmaceutical agent stability or release.

In the method of the first aspect of the invention, the nature (such as viscosity) of the spraying composition and duration of spraying can affect the orientation of layers within the mould. For example, compositions with low viscosity may fill the mould laterally forming a layer with a substantially flat surface. Compositions with higher viscosity may “cling” more to the surface of the mould, forming a layer which follows the shape of the mould aperture.

The plurality of layers may be substantially parallel or perpendicular to the base of the mould. The plurality of layers may be substantially parallel to the shaft of the microneedle.

In the method of the fourth aspect of the invention, the nature (such as viscosity) of the spraying composition and duration of spraying can affect the distribution of spraying composition on the microneedle array between the microneedle tip, shaft and base of the array.

The layering approach enables the microneedle structure to be built with a high mass of material in the microneedle structure and adequate mechanical strength to penetrate the stratum corneum. The high mass is achieved by removal of solvent after each layer is applied before application of the next layer. This enables a high mass of solid material to be filled into the moulds. The mass achieved is increased by this method compared to the filling the moulds with a solution of material in one step. When the moulds are filled in one step with a solution which is subsequently dried, the remaining solid material is low due to the large percentage of liquid filled into the mould.

Subsequent layering may be used to produce a microneedle which comprises an outer layer and an inner layer, wherein the properties of the materials of the outer and inner layers are such that the outer layer dissolves before the inner layer following application of the microneedle to the skin of a subject.

The layering approach facilitates the production of microneedles with sharp tips, for example enabling material to be deposited in the tip of the mould by overcoming the surface tension effects observed by other filling methods which result in crooked tips. Sharp tips are beneficial for penetration into skin

The layering approach facilitates the production of microneedles with high mechanical strength, for example by providing an outer layer which fauns a tip of the microneedle which is made from a material of high mechanical strength, the remainder of the microneedle being made from a material of low mechanical strength.

The layering process may be used to produce a microneedle which comprises an outer layer and an inner layer, wherein (i) the outer layer is made from a material of low mechanical strength and the internal layer is made from a material of high mechanical strength; or (2) the inner layer is made from a material of low mechanical strength and the outer layer is made from a material of high mechanical strength.

The layering approach also facilitates the application of a protective coating to the microneedles to protect against destabilising effects such as humidity, oxygen and light (visible and UV).

For example, in a microneedle which comprises an outer layer and an inner layer, the composition of the outer layer may be such that it acts as a moisture barrier (e.g. phospholipid or stearic acid), a light barrier (e.g. 2-hydroxy-4-methoxybenzophenone), a barrier to oxidation or other degradative chemical reactions (e.g. alpha tocopherol), and/or a barrier for handling to protect the user from a toxic inner layer (e.g. carboxymethylcellulose) (for example when the inner layer comprises chemotherapy drugs).

Thus, the layering approach also facilitates the separation of incompatible materials within the microneedle matrix and/or microneedle array backing.

The layering process may be used to produce a microneedle having an outer layer and an inner layer, wherein the outer layer comprises a rapid dissolving excipient (e.g. trehalose) and the inner layer comprises a slow release excipient (e.g. high viscosity carboxymethylcellulose).

The layering process may be used to produce a microneedle having an outer layer and an inner layer, whereby the outer and inner layers contain incompatible materials that are not in contact with each other (e.g. two incompatible vaccines). The outer and inner layers may be separated by a third layer (e.g. lactose or carboxymethylcellulose) that divides the incompatible materials.

The layering process may be used to produce a microneedle having an outer layer and an inner layer, wherein the outer layer comprises a slow dissolving or porous or non-dissolving excipient (e.g. cross linked PVA with dispersed PEG molecules) and the inner layer comprises a composition for sampling body fluids (e.g. ion exchange resins), such that the outer layer acts as a cage for the material of the inner layer.

The layering process may be used to produce a microneedle having an outer layer and an inner layer, wherein the outer layer comprises an amphiphilic material (e.g. phospholipid or stearic acid).

### Pharmaceutical Agent

The pharmaceutical agent may comprise: a therapeutic substance, such as a drug; a diagnostic substance; or a vaccine.

The pharmaceutical agent may be thermolabile.

A vaccine composition may comprise a whole organism vaccine, comprising a live, killed or attenuated pathogen.

The vaccine composition may comprise a subunit of a pathogen, or a peptide or polypeptide derivable therefrom comprising one or more antigenic epitope(s). The vaccine composition may comprise a nucleotide sequence, such as an RNA or DNA sequence capable of encoding a peptide or polypeptide comprising one or more antigenic epitope(s).

The vaccine composition may comprise a vector capable of delivering such a nucleotide sequence to a target cell, such as a plasmid, a viral vector, a bacterial vector or a yeast vector.

The vaccine composition may comprise one or more viral vectors.

Viral vectors or viral delivery systems include, for example, adenoviral vectors, adeno-associated viral (AAV) vectors, herpes viral vectors, retroviral vectors (including lentiviral vectors) baculoviral vectors and poxvirus vectors.

The vaccine may comprise a recombinant poxvirus vector. The vaccine may comprise a non-replicating or replication impaired viral vector such as Modified vaccinia virus Ankara (MVA).

Examples of poxviruses include MVA, NYVAC, avipox viruses and the attenuated vaccinia strain M7.

Alternatively the vector may be based on an adenovirus.

The term “vaccine” encompasses both a prophylactic composition for the prevention of a disease and a therapeutic composition for the treatment of an existing disease.

To “treat” means to administer the vaccine to a subject having an existing disease in order to lessen, reduce or improve at least one symptom associated with the disease and/or to slow down, reduce or block the progression of the disease.

To “prevent” means to administer the vaccine to a subject who has not yet contracted the disease and/or who is not showing any symptoms of the disease to prevent or impair the cause of the disease (e.g. infection) or to reduce or prevent development of at least one symptom associated with the disease.

### Kits

The present invention also provides kits for use in the methods of the present invention.

The kit may comprise a composition for spraying into a mould or for spraying on to a microneedle.

The kit may also comprise a microneedle array or a microneedle-forming mould; spraying apparatus; and/or a backing and/or adhesive layer.

The kit may also comprise a pharmaceutical agent dispersed in of for dispersal in a composition either for forming a dissolvable microneedle or layer thereof or for spraying on to a microneedle to form a coating.

Kits of the present invention may also comprise instructions for use.

### Device

The present invention also provides a device comprising a microneedle or microneedle array according to the present invention.

The device may, for example be a dressing, bandage or plaster (e.g. band-aid) with microneedles attached.

Such a device may be, for example, used for conditions which cover the skin such as psoriasis.

### Subject

The subject may be a mammalian subject, in particular a human, or a domestic or livestock animal such as a cat, dog, rabbit, guinea pig, rodent, horse, goat, sheep, cow or pig. For veterinary applications, the patch may be applied to an area on the animal which has little hair, such as the inner ear, or it may be necessary to remove hair from the skin prior to patent application.

The subject may be a human subject, in particular suffering from or at risk from contracting a particular disease. The subject may be a child or and adult subject.

The subject may be a healthy subject, believed to be at risk from contracting a disease. Alternatively the subject may already have or have had a disease.

The subject may have been previously exposed to antigen either by contact with the pathogen (for example by infection) or by prior immunisation.

The invention will now be further described by way of Examples, which are meant to serve to assist one of ordinary skill in the art in carrying out the invention and are not intended in any way to limit the scope of the invention.

### EXAMPLES

### Example 1

### Microneedle Preparation

### Step 1. Microneedle Mould Preparation

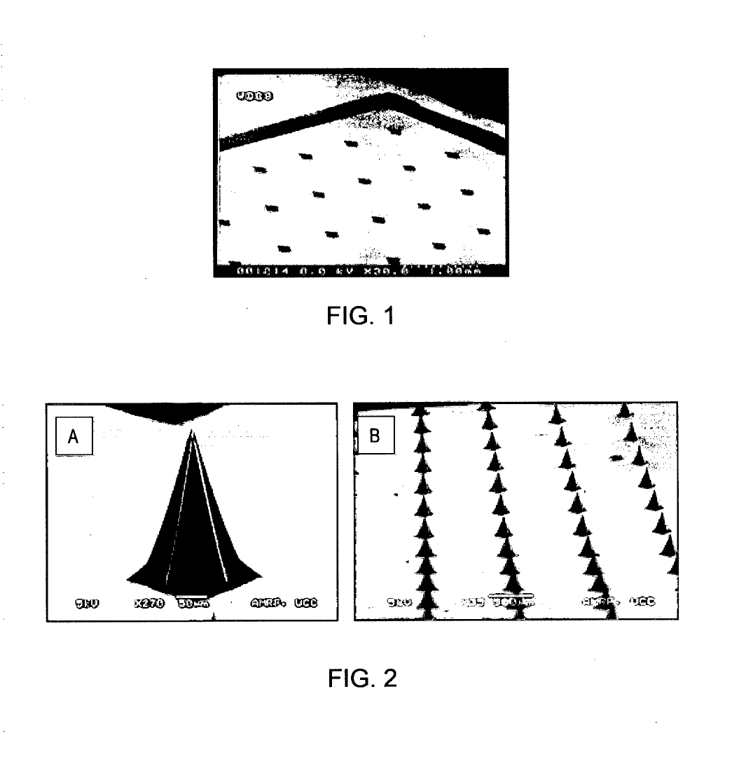

A master silicon microneedle array was manufactured by a silicon wet etching method (as described in U.S.2007/0134829A1 and Wilke el al (2005) Microelectronics Journal 36:650-656). Microneedle moulds were created from the master silicon microneedle array by pouring PDMS (polydimethylsiloxane) over the silicon array, curing at an elevated temperature (e.g. 100° C. for one hour) and then peeling the flexible PDMS mould from the master silicon array. FIG. 1 shows a scanning electron microscopy image of an inverted PDMS mould produced by this method.

### Step 2. Filling the PDMS Mould By Spraying A Mixture of Material For Microneedle Construction

A mixture, containing the material of construction, was prepared at a concentration of suitable viscosity for atomisation. Mixtures were atomised using a Schlick nozzle 970 S8, or equivalent, fitted with a 0.5 mm bore. An atomisation air setting of 2, gas pressure of 0.25 bars (air/nitrogen) and variable liquid input settings were used. The nozzle was positioned at a distance of 3.5 cm from the PDMS mould. The moulds were passed under the atomised spray. The duration of spraying varied, however in the majority of cases it was less than 1 second. Filled moulds were dried at room temperature and, where specified, in a low humidity desiccated environment.

The mass of material filled into the mould at each spraying step was dependent on the concentration of the mixture sprayed, the rate of liquid input and the duration of spraying. The moulds were filled in one or more spraying-drying operations. The process used to fill moulds in more than one spraying-drying operation was referred to as a ‘layering process’. During the layering process, the mould was passed under the spray and allowed to dry at room temperature for 5-30 mins and then passed under the spray again and dried. This process was repeated until the mould was filled. The eventual function of the microneedles determined the number of layers incorporated into the microneedles during the fabrication process.

The layering process enabled dense dissolvable microneedles with enhanced mechanical strength to penetrate the skin to be prepared. The layering process also enabled the preparation of dissolvable microneedles to be constructed with different materials organised in layers. There are a number of ways the layering process could be exploited for vaccine and drug delivery. For example (a) the application of a moisture barrier in the outer layer of the microneedles and a vaccine-trehalose mixture in the inner layers or (b) the application of a material of high mechanical strength for skin penetration at the tip of the microneedle and a protein-carbohydrate mixture composing the rest of the microneedle structure. Another example, (c) is composed of an outer layer(s) of drug mixed with a rapid dissolving excipient (e.g. sucrose, trehalose and raffinose) combined with inner layer(s) of drug mixed with a “slow” releasing excipient (e.g. PVA, PLGA, CMC). Such a system would enable rapid drug delivery upon initial penetration of skin with microneedles followed by sustained release of the same drug or a second compound over a prolonged time interval.

### Step 3. Application of Backing Layer And Removal From Mould

A backing layer of a solution of carboxymethylcellulose (CMC) (5% w/v) and glycerine (0.1% v/v) was then poured onto the filled mould and left to dry overnight. Drying in a low humidity, desiccated environment was also employed to increase the rate of drying. The microneedles were then removed from the mould. FIG. 2 shows CMC/glycerine microneedles with a CMC/glycerine backing prepared by the method described above.

### Example 2

### Preparation of Dissolvable Microneedles From A Range of Materials

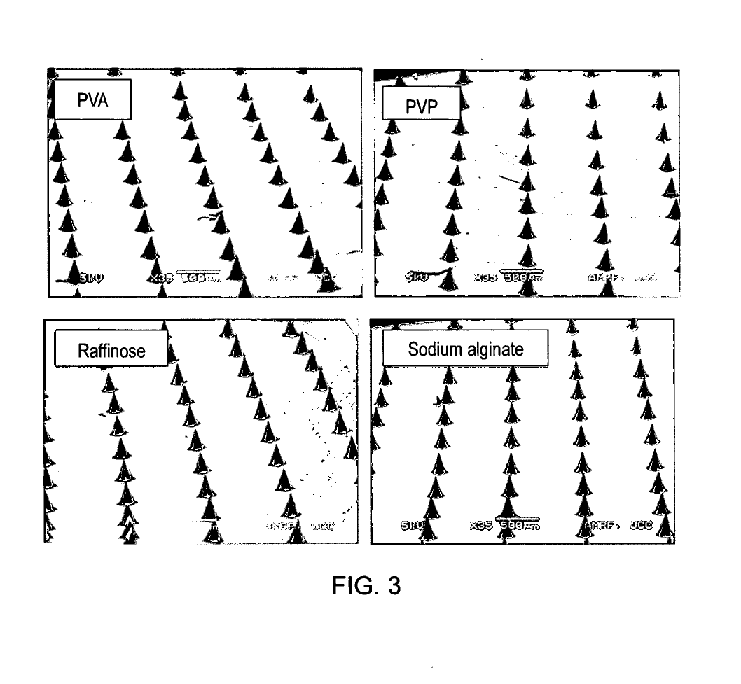

The method of preparation described in Example 1 can be used to prepare microneedles from a variety of dissolvable moieties such as polymers, cellulosics, sugars, polyols and alginic acid and its derivatives. FIG. 3 shows microneedles arrays prepared from polyvinyl alcohol (PVA), polyvinyl pyrrolidone (PVP), raffinose and sodium alginate.

Microneedles were prepared from 1% w/v aqueous solutions of PVA, PVP and raffinose and 0.35% w/v sodium alginate. Solutions were filter sterilised through a 0.4 m filter prior to atomisation when live virus vaccines were incorporated in the formulation. Solutions were atomised using a Schlick nozzle 970 S8 fitted with a 0.5 mm bore, an atomisation air setting of 2 and gas pressure of 0.25 bars (air/nitrogen). The nozzle was positioned at a distance of 3.5 cm from the PDMS mould. The moulds were passed under the spray. Spray times of less than 1 second were used. After spraying, each layer was left to dry for 5 minutes. Five layers of solution where applied. A backing layer of a solution of low viscosity sodium carboxymethylcellulose solution (5% w/v) and glycerine (0.1% v/v) was then applied. The microneedle array was left to dry overnight at room temperature prior to removal from mould. FIG. 3 shows microneedles prepared by this method.

### Example 3

### Preparation of Microneedles Comprising Layers of Different Materials

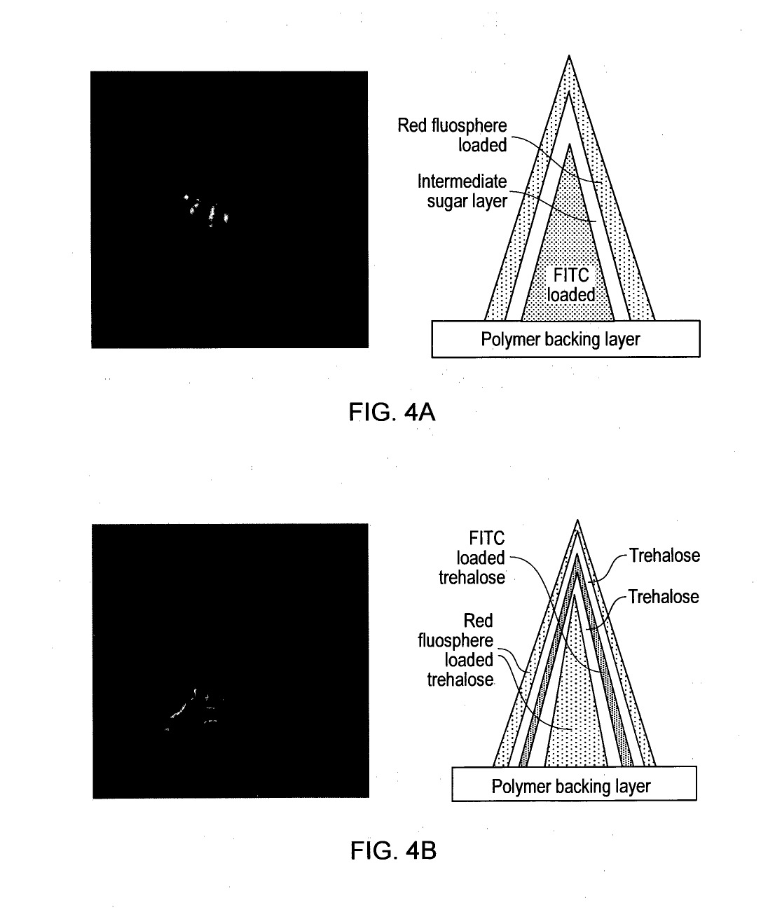

The method of preparation described in Example 1 can be used to prepare microneedles composed of distinct layers of material. To prove this claim, layers of material were sprayed into the PDMS moulds using fluorescent materials that could be identified by fluorescent microscopy; red fluospheres and green fluorescein. FIG. 4 shows two ways these layers can be organised.

Microneedles in FIG. 4A were prepared by an initial rapid spray (<1 second) of a mixture of red fluospheres loaded in an aqueous solution of trehalose (1% w/v), followed by a drying step, followed by a 5 second spray of a mixture of fluoroscein loaded in an aqueous solution of trehalose (1% w/v) followed by a drying step. Each layer was left to dry for 5 minutes prior to application of the next layer. The backing layer of a solution of CMC (5% w/v) and (0.1% v/v) glycerine was applied using a syringe. The microneedle array was left to dry overnight at room temperature, in a dark environment prior to removal from the mould. All mixtures were atomised using a Schlick nozzle 970 S8 fitted with a 0.5 mm bore, an atomisation air setting of 2 and gas pressure of 0.5 bars (air/nitrogen). The nozzle was positioned at a distance of 3.5 cm from the PDMS mould.

Microneedles in FIG. 4B were prepared by an initial 1 second spray of a mixture of red fluospheres loaded in an aqueous solution of trehalose (1% w/v), followed by a drying step, followed by a 1 second spray of an aqueous solution of trehalose (1% w/v), followed by a drying step, followed by a final 2 second spray of a mixture of fluoroscein loaded in an aqueous solution of trehalose (1% w/v) followed by a drying step. Each layer was left to dry for 5 minutes prior to application of the next layer. The backing layer of a solution of CMC (5% w/v) and (0.1% v/v) glycerine was applied. The microneedle array was left to dry overnight at room temperature, in a dark environment prior to removal from the mould. All mixtures were atomised using a Schlick nozzle 970 S8 fitted with a 0 5 mm bore, an atomisation air setting of 2 and gas pressure of 0.25 bars (air/nitrogen). The nozzle was positioned at a distance of 3.5 cm from the PDMS mould.

Increased or decreased duration of spray can increase the thickness of the layer formed. Variations in the atomisation air setting, the air pressure and the distance from the nozzle tip to the mould can also alter the structure of the layers formed. Composition of the sprayed mixture can also influence layer formation. For example, mixture interfacial tension and viscosity can influence the distribution and flow of mixture within the moulds, while vapour pressure can influence the rate of drying within the mould.

### Example 4

### Preparation of Carbohydrate Microneedles Containing An Outer Hydrophobic Coat

The method of preparation described in Example 1 can be used to prepare carbohydrate microneedles with an outer hydrophobic layer which would reduce the uptake of moisture in humid environments. Uptake of moisture in humid conditions has been reported to be a cause of instability for carbohydrate microneedle arrays (Donnelly et al (2009) Drug Dev Ind Pharm 35: 1242-54).

An outer layer of a poorly-soluble, amphiphilic material (Lipoid PG 18:0/18:0) was sprayed into the PDMS microneedle mould. After this layer was dried, the mould was filled by spraying a trehalose solution. Due to its amphiphilic nature, it was expected that the hydrophobic part of dissolved Lipoid PG 18:0/18:0 molecules would orient towards the PDMS and the hydrophilic part towards the trehalose material. Such an arrangement would leave the microneedles coated with a thin hydrophobic coat when removed from the mould.

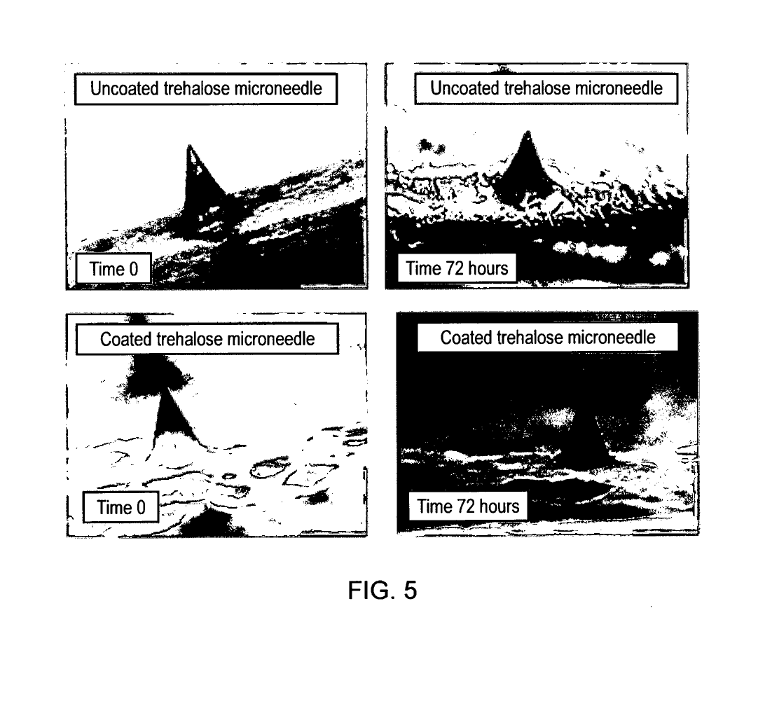

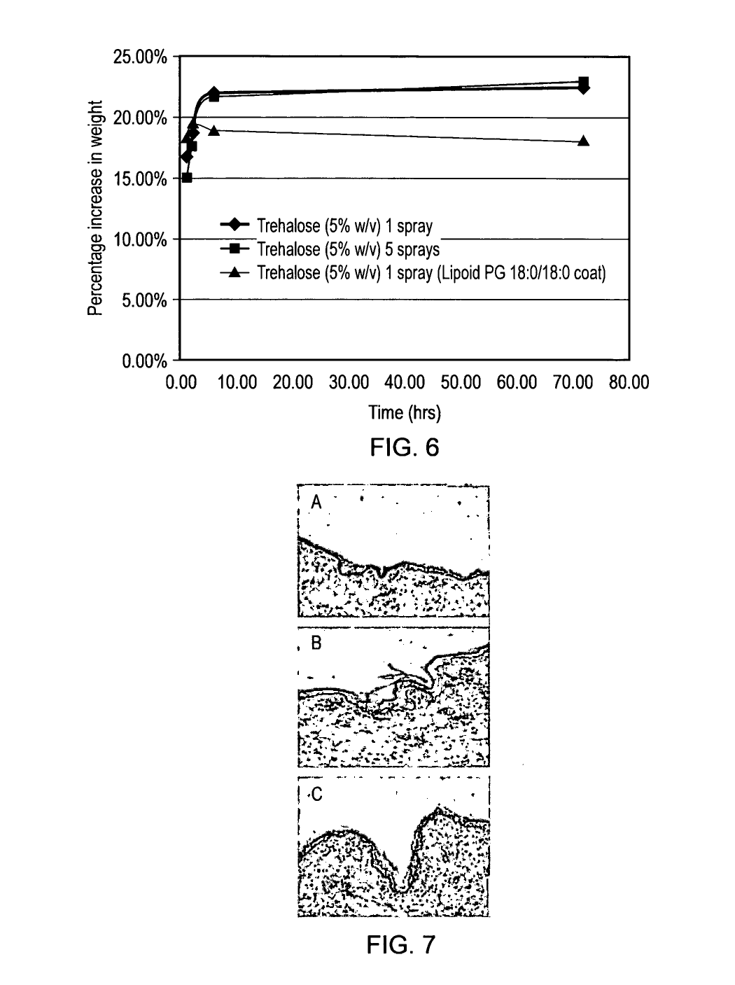

FIG. 5 shows microscope images of trehalose microneedles, prepared with and without a Lipoid PG 18:0/18:0 coating, after preparation and after 3 days storage exposed to an environment of 40° C. and 75% relative humidity. FIG. 6 shows the weight increase due to moisture uptake by these microneedles during storage. It can be observed that after 3 days the microneedles with the Lipoid PG 18:0/18:0 coat retained their structure, while a loss of sharp edges was apparent in the trehalose microneedles without the Lipoid PG 18:0/18:0 coat. The weight increase due to moisture uptake was significantly reduced for Lipoid PG 18:0/18:0 coated microneedles (FIG. 6).

### Example 5

### Demonstration That the Microneedles Are Capable of Penetrating the Stratum Corneum And Epidermis

Penetration of the stratum corneum and epidermis was investigated using ex vivo pig skin and examining for the generation of stratum corneum ruptures and epidermal breaches following application of the microneedles.

A variety of microneedles (described in Example 2) were applied to the pig skin ex vivo. Skin was prepared according to Coulman et al., (2006, Curr Drug Delivery 3:65-75). Microneedles were applied for 30 seconds and then removed. The tissue was then snap frozen and cryo-sectioned into 10 μm sections. Samples were H&E stained and examined by light microscopy. A number of features were observed in the skin sections and classified into the following categories; indentations without rupture, stratum corneum ruptures and epidermal breaches. FIG. 7 shows examples of these categories; a typical indentation without rupture (A), a stratum corneum rupture (B) and epidermal breach (C).

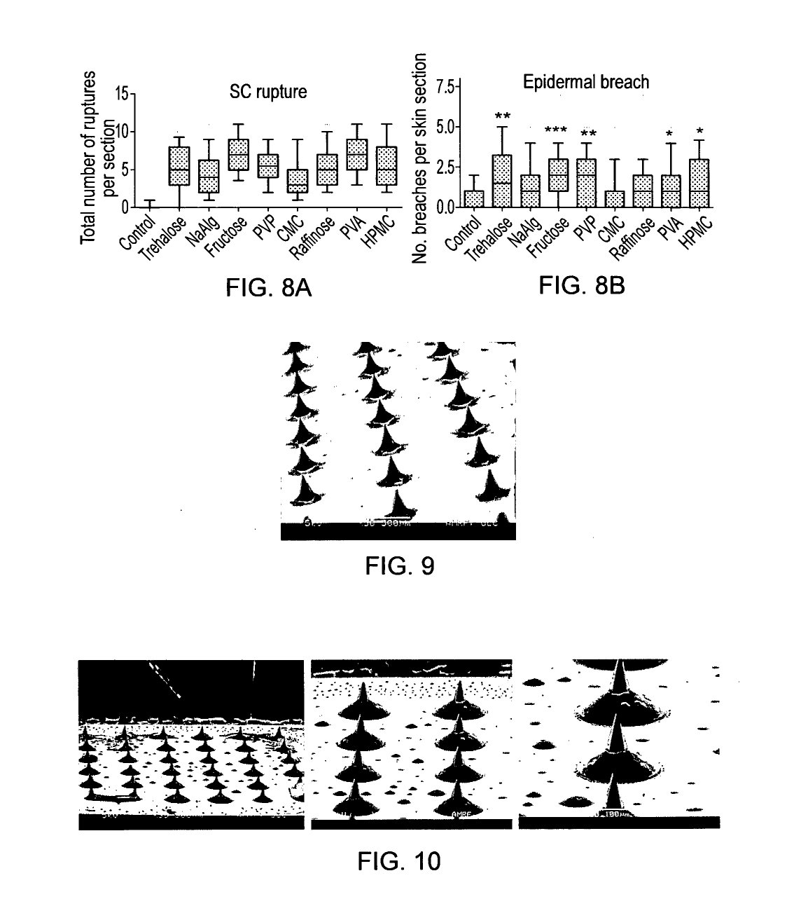

Approximately 450 fields of view were examined for each microneedle type or for control untreated skin. Within each section, five fields of view were examined and the total number of SC ruptures, indentations breaches etc per field of view was determined. Control sections of skin, without microneedle application, were used to determine baseline levels of SC rupture etc. FIG. 8 shows the total number of ‘stratum corneum ruptures’ (A) and ‘epidermal breaches’ (B) per section examined respectively. There was a significantly higher number of stratum corneum ruptures for all microneedle treated samples compared to control sample (p<0.0001) (FIG. 9A). A significantly higher number of epidermal breaches for microneedle treated samples compared to control samples was observed when microneedles were fabricated from trehalose, fructose, PVP, PVA and HPMC. This demonstrates that the choice of material to fabricate the microneedle impacts on the capacity of the microneedle array to penetrate into skin, an important factor for drug or vaccine delivery by microneedles. Therefore, microneedles constructed from trehalose, fructose, PVP, PVA and HPMC but not from CMC, raffinose or sodium alginate (NaAlg), are suitable for drug and vaccine delivery.

### Example 6

### Preparation of Silicon Microneedles Containing An Outer Carbohydrate Coat Containing Biopharmaceutical Molecules And Vaccines

Silicon microneedle arrays were fabricated by a wet-etch method according to Wilke (see above). A mixture, containing the material of coating, was prepared at a concentration of suitable viscosity for atomisation. Mixtures were atomised using a Schlick nozzle 970 S8, or equivalent, fitted with a 0.5 mm bore. An atomisation air setting of 2, gas pressure of 0.25 bars (air/nitrogen) and variable liquid input settings were used. The nozzle was positioned at various distances from the silicon microneedle array. The microneedle arrays were passed under the atomised spray. The duration of spraying varied (see examples doc). Including a surfactant, such as Tween20 or lutrol in the mixture resulted in the coating being located on the base of the microneedle array and on the needle shaft. Coated microneedle arrays were dried at room temperature and, where specified, in a low humidity desiccated environment.

FIG. 9 shows scanning electron microscopy images of Carboxymethylcellulose sodium (3% w/v) and Tween 80 (1% v/v) aqueous solution spray dried onto a silicon wafer. The distance from the nozzle to the wafer was 6 cm. The liquid input was at a rate of 1.5 ml/min and compressed air pressure 1 bar. The coating thickness obtained where approx 5 microns

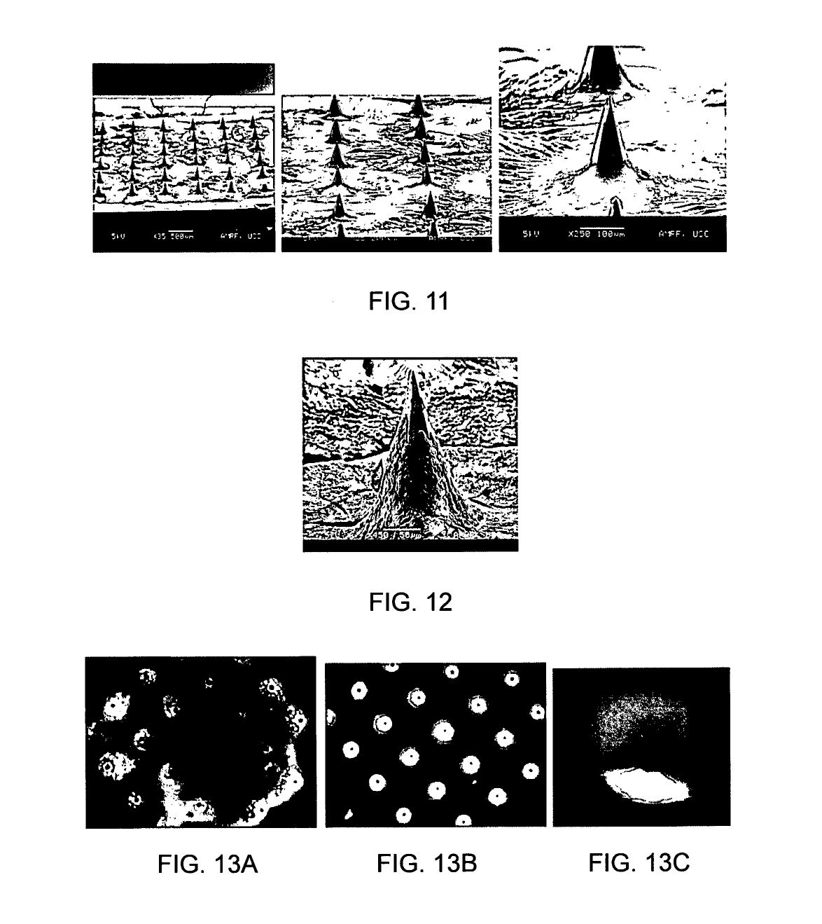

Altering the rate of liquid input into the nozzle determines the coating pattern. Slow spraying (at 1 ml/32.5 seconds) results in coating the material around the needle shaft only, as shown in FIG. 10. In contrast, increasing the rate of spraying to 1 ml/5.5 seconds results in coating the microneedle shaft and the base of the microneedle array, as shown in FIG. 11.

Altering the composition of the mixture also affects the coating pattern. Addition of carboxymethylcellulose (CMC 1% w/v) which increases the viscosity of the trehalose/tween formulation, results in coating the microneedle shaft and the base of the microneedle array, as shown in FIG. 12.

As shown in FIG. 13, spray coating (B and C) results in even distribution of the mixture around the microneedles compared to pippetting the mixture onto the microneedle array (A).

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in microneedle technology or related fields are intended to be within the scope of the following claims.

1. A method for fabricating a microneedle which comprises the steps of spraying a composition into a mould, drying the composition and removing the dried composition from the mould, thereby forming a microneedle that, when applied to the skin of a subject, pierces the stratum corneum to access the underlying tissue of the subject.

2. The method according to claim 1, wherein the composition forms a dissolvable material following drying, such that when the microneedle is applied to the skin of a subject it dissolves.

3. The method according to claim 2, wherein the composition comprises an agent dispersed in a solution which forms a dissolvable material following drying, such that when the microneedle is applied to the skin of a subject it dissolves, causing delivery of the agent into the underlying dermal tissue of the subject.

4. The method according to claim 1, wherein the composition is dried at ambient temperature.

5. The method according to claim 1, which comprises a plurality of successive spraying and drying steps to create a plurality of layers.

6. (canceled)

7. The method according to claim 5, wherein the successive spraying and drying steps involve application of a plurality of different compositions which comprise different agents or different materials.

8-10. (canceled)

11. The method according to claim 3, wherein the agent is thermolabile.

12-14. (canceled)

15. The method according to claim 1, for fabricating a microneedle comprising an outer layer and an inner layer, in which method the outer layer is fabricated by spraying a composition into a microneedle mould.

16. (canceled)

17. The microneedle fabricated by a method according to claim 15.

18. The microneedle according to claim 17, wherein the outer layer dissolves before the inner layer following application of the microneedle to the skin of a subject.

19. The microneedle according to claim 17, wherein the outer layer is a moisture barrier; a light barrier; a barrier to oxidation or other degradative chemical reactions; and/or a barrier for handling to protect the user from a toxic inner layer.

20. The microneedle according to claim 17, wherein the outer layer comprises a rapid dissolving excipient and the inner layer comprises a slow release excipient.

21. The microneedle according to claim 17, which comprises an outer layer and an inner layer, whereby the outer and inner layers contain incompatible materials that are not in contact with each other.

22. The microneedle according to claim 17, whereby the outer and inner layers are separated by a third layer that divides the incompatible materials.

23. The microneedle according to claim 17, wherein the outer layer forms a tip of the microneedle which is made from a material of high mechanical strength and the remainder of the microneedle is made from a material of low mechanical strength.

24. The microneedle according to claim 17, wherein the outer layer is made from a material of low mechanical strength and the internal layer is made from a material of high mechanical strength.

25. The microneedle according to claim 17, wherein the outer layer comprises a slow dissolving or porous or non-dissolving excipient and the inner layer comprises a composition for sampling body fluids, such that the outer layer acts as a cage for the material of the inner layer.

26-29. (canceled)

30. The method according to claim 1 which comprises the step of applying a backing layer to the mould, once filled.

31. (canceled)

32. (canceled)

33. The method according to claim 1 for making a microneedle array comprising a plurality of microneedles which comprises the step of spraying a composition into a mould which comprises a plurality of microneedle-forming apertures.

34-36. (canceled)

37. A method for coating a microneedle which comprises the steps of spraying a composition onto a microneedle and drying the composition at an ambient temperature, thereby forming a coated microneedle that, when applied to the skin of a subject, pierces the stratum corneum to deliver the sprayed material to the underlying tissue of the subject.

38-39. (canceled)
